(19) United States
(12) Patent Application Publication
(10) Pub. No.: US 2011/0086003 A1
(43) Pub. Date: Apr. 14, 2011

(54) STABILIZATION OF HYDROPHOBIC PROTEIN THERAPEUTIC AGENTS

(75) Inventors: Bernd Otto, Hannover (DE); Florian Battermann, Hannover (DE); Rudiger Kohl, Hannover (DE)

(73) Assignee: STIFTUNG TIERARZTLICHE HOCHSCHULE HANNOVER, Hannover (DE)

(21) Appl. No.: 12/738,818

(22) PCT Filed: Oct. 7, 2008
(86) PCT No.: PCT/EP08/08450

§ 371 Date: Dec. 28, 2010

(30) Foreign Application Priority Data
Oct. 19, 2007 (DE) 10 2007 050 165.1

(51) Int. Cl. A61K 38/00; A61K 38/21; A61K 38/18; A61K 38/20; A61K 38/19; A61K 38/28; A61K 38/27; A61P 3/10; A61P 7/06; A61P 31/12; A61P 35/00; A61P 37/02

(52) U.S. Cl. 424/85.2; 514/1.1; 424/85.5; 424/85.6; 424/85.7; 514/7.7; 424/85.1; 514/5.9; 514/11.3; 514/6.9; 514/13.5; 514/3.7; 514/19.3

(57) ABSTRACT

The present invention relates to a stabilized solution comprising a hydrophobic protein, a method for the production thereof, and the stabilizing solution as a pharmaceutical, and the use thereof.

The present invention relates to a stabilized solution comprising a hydrophobic protein, a method for the production thereof, as well as to the stabilized solution as a drug, and the use thereof.

Hydrophobic proteins are used commonly in human medicine as therapeutic agents. However, the problem with this use is that the hydrophobic protein therapeutic agents, in the absence of special stabilizers, form aggregates as a function of the concentration, pH, and the ionic strength of the reagents during the workup, formulation and storage, where these aggregates contain inactive and immunogenic protein. To circumvent this problem, human serum albumin (HSA) has been used in the past in the culturing, workup and formulation. Because the use of natural HSA (which originates from human plasma) increases the risk of viral contamination, and the use of recombinant HSA is not economic for financial reasons, HSA is no longer used today. Moreover, HSA is no longer approved by the regulatory authorities for the above mentioned reasons and because of the fact that the presence of HSA makes quality control more difficult in regard to the degree of purity.

Carbohydrates and detergents, such as, polysorbates, are used today as an HSA replacement; however, they are disadvantageous, because of their tendency to form immunogenic aggregates. Alternatively, amino acids are used with carbohydrates for stabilization of the protein therapeutic agents; however, they require the addition of high concentrations of arginine (up to 50 mg/mL), and still do not yield satisfactory results with regard to the stabilization. In addition, the addition of high arginine concentrations results in an extremely low pH of the therapeutic agents that have been stabilized in this way, so that, for example, in the case of parenteral administration, the proteins being used aggregate, and thus produce noxious agents at the puncture sites.

The problem of the present invention therefore is to produce a stabilized solution of a hydrophobic protein, where the risk of viral contamination does not exist, and whose administration, while being compatible from the point of view of health, does not induce any adverse reactions in the patients. Moreover, when they are stored, no loss of activity or aggregate formation should occur, and the manufacture of the solution should be simple and cost effective. This problem is solved by the stabilized solution, the method for the preparation thereof, and its use according to the present independent claims. Advantageous embodiments are indicated in the dependent claims.

According to the invention, the term hydrophobic protein denotes a protein that is characterized by a high proportion of hydrophobic amino acids and a low solubility. For example, a natural or recombinant human interferon β has a proportion of 40% hydrophobic amino acids. Low solubility denotes a solubility <0.3 mg/mL. In the present invention, as hydrophobic proteins, proteins are used that bind with high affinity to the given receptor proteins via hydrophobic sites (binding sites). In the present invention, it is preferred to use, as hydrophobic proteins, natural or recombinant interferon alpha, β, gamma, erythropoietin, interleukin II, G-CSF, colony stimulating factors, insulin, growth hormone, cytokines and/or mixtures thereof. Natural or recombinant human interferon β is mentioned as preferred.

The polyvalent alcohols used for stabilization are alcohols that have at least two OH groups, where the OH groups are arranged in such a way that the alcohols have a hydrophilic and a hydrophobic region. Moreover, the polyvalent alcohols being used should not have any properties harmful to health, and if applicable they should be approved for parenteral administration. Examples of polyvalent alcohols that are suitable for the invention are short-chained, polyvalent alcohols as well as oligomers. It is preferable to use propylene glycol, glycerol, butylene glycol, pentanediol and/or mixtures thereof. It is particularly preferable to use propylene glycol and glycerol. The concentration of the alcohol to be used is a function of the degree of hydrophobicity of the hydrophobic protein. Thus, in rhuinterferon β, for example, 1-60% polyvalent alcohols are used for stabilization.

According to the invention, the stabilized solution contains polyvalent alcohols at a concentration of 1-60 vol %, preferably, 1-20 vol %, particularly preferably 5-15 vol %, with respect to the total volume of the stabilized solution.

The stabilized solution contains the hydrophobic proteins according to the solution at a concentration of 10-300 μg/mL, preferably 10-200 μg/mL, particularly preferred 80-120 μg/mL, with respect to the total volume of the stabilized solution.

The hydrophobic protein and the polyvalent alcohol are usually present in the stabilized solution at a ratio from 1:1000 to 1:10,000, preferably from 1:200 to 1:2000, particularly preferably from 1:500 to 1:1500.

According to the invention, the term neutral pH denotes a pH of 6-8, preferably a pH of 6.5-7.5, particularly preferably 6.8-7.2.

Besides the polyvalent alcohols, the stabilized solution according to the invention can contain other excipients and additives. Pertinent examples are sugar alcohols, such as, for example, mannitol and/or sorbitol.

Suitable buffer media, which the stabilized solution according to the invention may optionally contain, are buffered salt solutions with neutral pH. It is preferred to use an Na phosphate buffer as the buffer medium. Tris buffers and Hepes buffers could also be used.

The stabilized solution can also be lyophilized. The corresponding lyophilisate must be dissolved in an appropriate buffer medium before application.

An additional object of the present invention is a method for the preparation of a stabilized solution according to the invention, which is characterized by the successive use of polyvalent alcohols and the maintenance of a neutral pH during the individual processing steps. The preparation of a stabilized solution according to the invention, which comprises a hydrophobic protein according to the method of the invention, is characterized in that the workup of the hydrophobic protein occurs via at least one chromatographic step, preferably three chromatographic steps, in which at least one polyvalent alcohol is added to the cell culture supernatant obtained from cell culturing and/or from the eluates obtained from the respective chromatography steps and/or the elution agents, and the individual steps are carried out at a neutral pH. Optionally, a polyvalent alcohol can be added already during the culturing (expression and secretion) of the cells that produce the given hydrophobic protein.

The term “workup” in the present context denotes the concentration of the given hydrophobic protein with simultaneous separation of secreted host cell proteins and foreign protein portions, which result, for example, from the protein additives already present in the media mixture used for cell culturing. As preferred separation methods leading to a high degree of purification of the hydrophobic protein, chromatographic methods should be mentioned.

As preferred chromatography methods for the individual chromatography steps according to the invention, affinity chromatography and/or gel filtration chromatography methods should be mentioned. It is preferred to use affinity column chromatography columns whose column matrices form hydrophobic and/or metal chelate complexes with the hydrophobic proteins. For gel filtration column chromatography, columns are used that separate the proteins based on their different sizes.

The order used in the claims for the individual chromatography steps is not binding and can be varied. Moreover, it is possible to carry out only one chromatography step.

Suitable elution agents for carrying out affinity chromatography, in which the column matrix develops hydrophobic interactions with the hydrophobic protein, are elution agents that eliminate the interactions between hydrophobic protein and hydrophobic affinity chromatography material, such as, for example, ethylene glycol, propylene glycol, glycerol and/or mixtures thereof. As columns to be used, one can consider Cibacron Blue Sepharose, poly(A), glutathione, ConA, heparin Sepharose columns and/or combinations of these columns, preferably a Cibacron Blue Sepharose column, but also any other type of carrier material in the form of similar polymer materials.

Suitable elution agents for carrying out affinity chromatography, in which the column matrix forms metal chelate complexes with the hydrophobic protein, are salts that are capable of changing the pH and/or imidazole. As columns to be used, one can consider zinc chelate, nickel chelate, chromium chelate columns and/or combinations of these columns, preferably zinc chelate (chelating Sepharose).

Suitable elution agents for carrying out gel filtration chromatography are elution agents that are capable of separating the given hydrophobic protein in a molecular weight range of 5-200,000 Da, as a function of the column material. The column material should be chosen in such a way that only weak hydrophobic interactions are developed between the column material and the hydrophobic protein. As columns to be used, one can consider P75, S-100, S-200, Biogel P30 columns, particularly a p75 column.

An additional object of the present invention is the stabilized solution according to the invention as a drug, where this drug could comprise other excipients and additives, such as, for example, sugar alcohols, such as, for example, mannitol and/or sorbitol.

The administration of the drugs according to the invention occurs parenterally, for example, intramuscularly, subcutaneously, or intravenously. The drug according to the invention contains the given hydrophobic protein in each case in a quantity of 5-300 μg/dose, preferably 5-100 μg/dose, where the injection frequency is a function of the hydrophobic protein being used and the indication.

The drug according to the invention can comprise other drugs besides the stabilized solution according to the invention. As just an example, the combination of human interferon β with glatiramer acetate (Copaxone) is mentioned.

Another object of the present invention is the use of the stabilized solution according to the invention for the preparation of a drug to treat diabetes, anemia, viral infections, tumors, autoimmune diseases and/or combinations of these diseases.

As just an example, a possible, non-limiting embodiment of the present invention is explained below using human interferon β (IFN-β).

% data given below always refer to volume percents (V/V).

Culturing the Cells

As cells, CHO cells (Chinese hamster ovarian cells) are used. The cells are cultured in a media mixture of 50% MEM alpha and 50% CHO S-SFM-II with 1% MEM vitamin and 1% glycerol. No effect on cell vitally caused by glycerol was observed.

1st Column Step: Cibacron Blue Sepharose Column

Adjusting the cell culture supernatant to 15% ethylene glycol and 1 M NaCl in a cell culture buffer (Na carbonate, carbonic acid buffer) pH 7.2.

Elution of the column with 0.02 M phosphate buffer pH 7.2 with 1 M NaCl and 60% ethylene glycol.

The added ethylene glycol is separated completely in the subsequent steps. However, it is also possible to omit ethylene glycol entirely and to use propylene glycol instead.

2nd Column Step: Zinc Chelate (Chelating Sepharose)

Adjusting the eluate of Cibacron Blue Sepharose (Blue Sepharose eluate) to 0.02M phosphate buffer pH 7.0 with 0.5 M NaCl and 30% ethylene glycol.

After the application, washing with a washing buffer containing 10% glycerol and 0.5 M NaCl in a 0.02 M phosphate pH 7.0.

Elution with a phosphate buffer (0.02M) with 0.5 M NaCl, 10% glycerol and 0.1 M imidazole.

3rd Column Step: Gel Filtration in a P75 16 60

Injection of the eluate of the chelate column; followed by a run in the buffer.

0.02 M phosphate buffer, 10% propylene glycol, 0.5 M NaCl pH 6.8.

A subsequent immediate stabilization with HSA is not required.

Overview of the results of the workup methods

| | Workup according to the invention |
|---|---|
| | (without HSA with polyvalent alcohols) |
| | Yield (total) (%) |
| Cell culture supernatant | 100 |
| Cibacron Blue Sepharose | 85 (85) |
| Chelating Sepharose | 81 (72) |
| Gel filtration | 77 (53) |

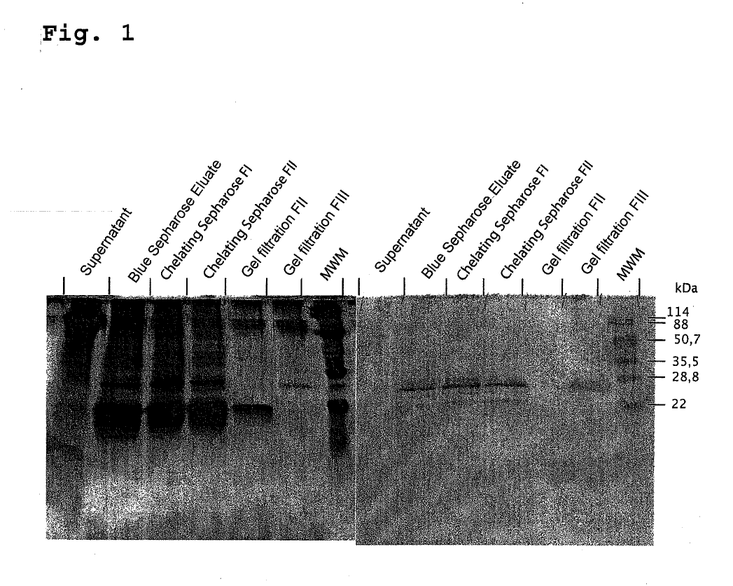

FIG. 1 shows an overview of the workup across all of the columns both using silver staining and also using specific Western blot.

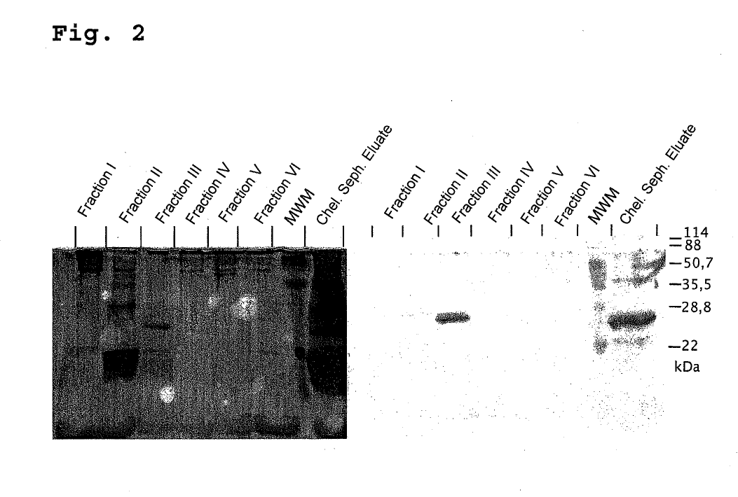

FIG. 2 shows the silver staining and Western blot of the individual fractions of a gel filtration. As one can see in FIG. 2, only fraction III of the gel filtration contains the interferon beta. The good separation between interferon (fraction III) and foreign proteins is noteworthy. As reference, the eluate of the chelating Sepharose was used.

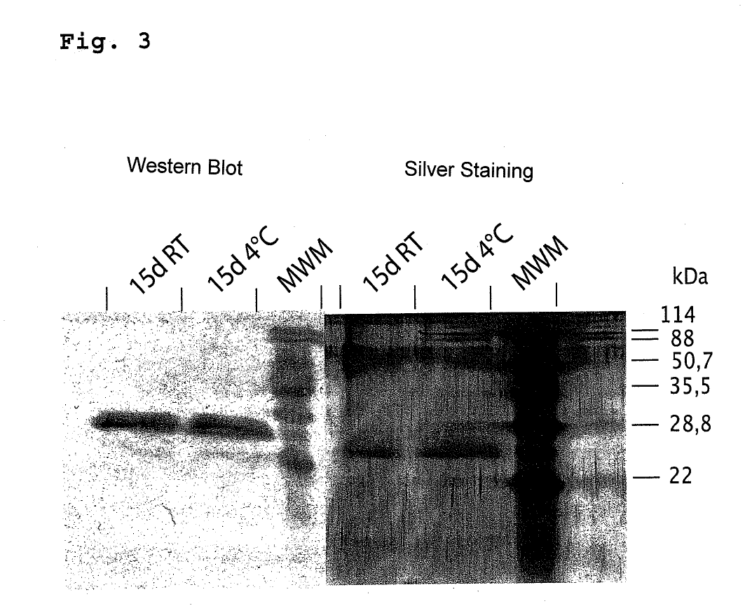

FIG. 3 gives an overview of the stability test:

To check the stability of the formulation, the product of gel filtration was concentrated and sterile filtered. A sample was incubated for 15 days at room temperature; an aliquot at 4° C. was used for comparison. No degradation products or degradation was observed. The activity of the sample, in comparison with the 4° C. control and the value measured before the incubation, was identical within the accuracy of the measurement. This confirms that hydrophobic proteins alone can be stabilized by at least one polyvalent alcohol.

FIG. 3 shows the stability test of the gel-filtration-purified and concentrated product. One can see clearly that there is no detectable degradation after 15 days at room temperature.

1. A stabilized solution comprising a hydrophobic protein and at least one polyvalent alcohol for stabilization of the solution, where the solution has a neutral pH and is free of human serum albumin.

2. The stabilized solution according to claim 1, wherein, besides containing at least one polyvalent alcohol for stabilization, the solution contains no other substances for stabilization.

3. The stabilized solution according to claim 1, wherein the hydrophobic protein is a human or animal hydrophobic protein.

4. The stabilized solution according to claim 1, wherein it contains at least one polyvalent alcohol as a stabilizer.

5. The stabilized solution according to claim 4, wherein the polyvalent alcohol has at least two OH groups, a hydrophilic region and a hydrophobic region.

6. The stabilized solution according to claim 5, wherein the polyvalent alcohol is selected from the group consisting of ethylene glycol, propylene glycol, glycerol, butylene glycol, pentanediol and/or mixtures thereof.

7. The stabilized solution according to claim 1, wherein the stabilized solution contains the polyvalent alcohol at a concentration of 1-60 vol %, with respect to the total volume of the stabilized solution.

8. The stabilized solution according to claim 1, wherein the hydrophobic protein is selected from the group consisting of natural or recombinant human interferon alpha, β, or gamma, erythropoietin, interleukin II, G-CSF, colony stimulating factors, insulin, growth hormone, cytokine and mixtures thereof.

9. The stabilized solution according to claim 1, wherein the stabilized solution contains the hydrophobic protein at a concentration of 10-300 μg/mL with respect to the total volume of the stabilized solution.

10. The stabilized solution according to claim 1, wherein the stabilized solution contains the hydrophobic protein and the polyvalent alcohol at a ratio from 1:1000 to 1:10,000 (hydrophobic protein to polyvalent alcohol).

11. The stabilized solution claim 1, wherein the stabilized solution contains the natural or recombinant interferon β and the polyvalent alcohol at a ratio from 1:500 to 1:1500 (natural or recombinant human interferon β to polyvalent alcohol).

12. The stabilized solution according to claim 1, wherein the stabilized solution also contains a buffer medium.

13. The stabilized solution according to claim 12, wherein the buffer medium is a buffered salt solution.

14. The stabilized solution according to claim 1, wherein the pH of the solution is in a range of pH 6.0-8.0.

15. The stabilized solution according to claim 1, wherein the stabilized solution consists of at least one hydrophobic protein, at least one polyvalent alcohol for stabilization, and at least one buffer medium.

16. A method for the preparation of a stabilized solution according to claim 1, wherein a cell culture supernatant containing the hydrophobic protein is worked up without the use of a human serum albumin in at least one chromatography step, where the cell culture supernatant and/or the given eluates from the given chromatography steps and/or the given elution agents have a neutral pH and contain at least one polyvalent alcohol.

17. The method according to claim 16, wherein the at least one chromatography step is affinity chromatography step and gel filtration chromatography step.

18. The method according to claim 16, comprising the following steps:
a) optionally culturing the given hydrophobic protein producing cells in a media mixture that contains at least one polyvalent alcohol at a neutral pH,
b) working up the cell culture supernatant that contains the hydrophobic protein by affinity column chromatography, utilizing a column matrix that develops hydrophobic interactions with the hydrophobic protein, and/or
c) working up the cell culture supernatant or the eluate obtained from step b) by affinity column chromatography utilizing a column matrix that forms metal chelate complexes with the eluate, and/or
d) working up the cell culture supernatant or of the eluate obtained form step b) or c) by gel filtration column chromatography, and
e) of stabilizing the hydrophobic proteins.

19. The method according to claim 18, wherein in step b) a Cibacron Blue Sepharose and/or in step c) a zinc chelate column is utilized.

20. The method according to claim 18, wherein in step b) the cell culture supernatant is adjusted with a solution that contains at least one polyvalent alcohol to a neutral pH and then eluted with an elution agent from the column.

21. The method according to claim 18, wherein in step c) the cell culture supernatant or the eluate obtained from step b) is adjusted with a solution that contains at least one polyvalent alcohol to a neutral pH, optionally washed, after the application to the column matrix, with a neutral washing buffer that contains at least one polyvalent alcohol, and then eluted with an elution agent from the column.

22. The method according to claim 18, wherein the gel filtration of the cell culture supernatant or of the eluate obtained from step b) or c) occurs at a neutral pH, and an eluent containing at least one polyvalent alcohol is used.

23. The method according to claim 18, wherein in step e) the cell culture supernatant or the eluate obtained from step b), c) or d) is stabilized with at least one polyvalent alcohol at a neutral pH.

24. The method according to claim 20, wherein the polyvalent alcohol is selected from the group consisting of ethylene glycol, propylene glycol, glycerol, butylene glycol, pentanediol and/or mixtures thereof.

25. The method according to claim 20, wherein the neutral pH is in a range of pH 6.0-8.0.

26. The stabilized solution according to claim 1, wherein the stabilized solution is a drug formulation.

27. The stabilized solution according to claim 26, wherein the drug formulation contains the hydrophobic protein in a quantity of 5-300 μg per dose.

28. The stabilized solution according to claim 26, wherein the drug is a lyophilisate.

29. A method of treating diabetes, anemia, viral infections, tumors, autoimmune diseases and/or combinations thereof, comprising administering an effective amount of the stabilized solution according to claim 1.

30. The stabilized solution according to claim 13, wherein the buffered salt is sodium phosphate.
